(19) United States
(12) Patent Application Publication
(10) Pub. No.: US 2005/0143603 A1
(43) Pub. Date: Jun. 30, 2005

(54) Process for the oxidation of unsaturated hydrocarbons

(76) Inventors: Gunther Bub, Marl (DE); Roland Fornika, Bonn (DE); Berthold Fischer, Herne (DE); Dimitrii Guschin, Bochum (DE); Torsten Meyer, Castrop/Rauxel (DE); William Sheldrick, Dusseldorf (DE)

Correspondence Address: SMITH MOORE LLP, P.O. BOX 21927, GREENSBORO, NC, 27420, US

(21) Appl. No.: 10/502,007

(22) PCT Filed: Jan. 16, 2003
(86) PCT No.: PCT/EP03/00407

(30) Foreign Application Priority Data
Jan. 17, 2002 (DE) 102 01 783.2

(51) Int. Cl. C08F 4/28; C07C 51/16

(52) U.S. Cl. 562/548; 526/227

(57) ABSTRACT

The invention relates to a process for the oxidation of unsaturated hydrocarbons, wherein an unsaturated hydrocarbon, an oxygen-containing oxidizing agent, a palladium complex as the catalyst containing a ligand of the formula (I) 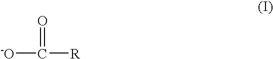 wherein R is a saturated, halogenated alkyl radical having from about 1 to about 20 carbon atoms, and optionally auxiliary substances in a liquid phase based on (α1) from about 10 to about 100 wt. % of a protic polar solvent and (α2) from 0 to about 90 wt. % of an aprotic polar solvent, the sum of components (α1) and (α2) being about 100 wt. %, at a temperature in a range from about 30 to about 300° C. under a pressure in a range from about 1 to about 200 bar, such that a liquid phase containing oxygen-containing hydrocarbons is obtained.

This application is a national stage application under 35 U.S.C. 371 of international application no. PCT/EP03/00407 filed Jan. 16, 2003, which is based on German Application no. DE 102 01 783.2, filed on Jan. 17, 2002, and claims priority thereto.

### BACKGROUND OF THE INVENTION

The invention relates to a process for the oxidation of unsaturated hydrocarbons, oxygen-containing hydrocarbons obtainable by this process, the liquid phase obtainable by this process, the oxygen-containing hydrocarbons obtainable by this process, chemical products comprising the oxygen-containing hydrocarbons, the use of these oxygen-containing hydrocarbons in chemical products, the use of acetic acid or of a salt of acetic acid in a process for the oxidation of unsaturated hydrocarbons, a process for the preparation of water-soluble or water-absorbent polymers, the water-soluble or water-absorbent polymers obtainable by this process, the use of a liquid phase for the preparation of water-soluble or water-absorbent polymers, a composite, a process for the production of a composite, a composite obtainable by this process, chemical products comprising the water-absorbent polymer or the composite, and the use of the water-absorbent polymer or of the composite in chemical products.

The oxidation of unsaturated hydrocarbons by atmospheric oxygen with the aid of heterogeneous or homogeneous catalysts is an industrially important process. Thus, for example, by the catalytic oxidation of propylene by air, acetone and acrylic acid are obtained as products which are employed in the synthesis of many products prepared on a large industrial scale. Nevertheless, the oxidation of unsaturated hydrocarbons by atmospheric oxygen as a rule leads to product mixtures. Thus, in the abovementioned oxidation of propylene by atmospheric oxygen, in addition to acetone and acrylic acid, other oxygen-containing products, for example acrolein, propionic acid, propionaldehyde, acetic acid, CO₂, acetaldehyde or methanol, are also obtained.

A number of processes have been described in the patent literature for the oxidation of olefins on an industrial scale, both in the gas phase and in the liquid phase. The selectivity of the oxidation of olefins by atmospheric oxygen depends above all on the reaction conditions and on the catalyst systems employed.

In order preferentially to achieve an allylic oxidation of unsaturated hydrocarbons, which in the case of propylene leads above all to acrylic acid as the main product, various processes and also various catalyst systems employed in these processes are described in the prior art. According to the current state of knowledge, of the noble metals Pd catalysts are preferred in order, for example, to convert propylene as selectively as possible into acrylic acid with a good yield in solvents under mild reaction conditions. Nevertheless, Pd catalysts also catalyse vinylic oxidation of unsaturated hydrocarbons, which leads above all to ketones, and in the case of propylene to acetone. The oxidation of α-unsaturated hydrocarbons on Pd can be directed, however, in the direction of an allylic oxidation by means of suitable electron-withdrawing ligands and by the choice of particular solvents (LYONS J. E., SULD G., HUS Ch. Y., “Homogeneous Heterog. Catal. Proc. Int. Symp. Relat.”, Homogeneous Heterog. Catal., 5th (1986): 117-138; TROST B. M., METZNER P. J., J. Am. Chem. Soc., 102 (1980): 3572; KETELEY A. D., BRAATZ J., Chem. Comm. (1968): 169).

Reduced Pd catalyses the oxidation of propylene to acrylic acid particularly selectively. For this, the reaction should be carried out with an excess of propylene (O₂/C₃H₆<1). Reduction of the Pd catalyst before the start of the reaction minimizes the formation of by-products by vinylic oxidation to acetone and acetic acid already at the start of oxidation (EP-A-145467, EP-A-145468 and EP-A-145469). Nevertheless, a disadvantage of the process described in these documents is the low catalyst output of a maximum of 0.038 g acrylic acid/g_{Pd}/hour.

In addition to allylic oxidation, however, it is also desirable to direct the oxidation of unsaturated hydrocarbons in the direction of a vinylic oxidation. Acetone can be prepared from propylene in this manner.

Industrially, acetone is prepared, for example, in co-production with phenol by oxidation of cumene or by dehydrogenation of isopropyl alcohol. The process mentioned first has the disadvantage of a stoichiometric production of a by-product (phenol), while in the older second process the dehydrogenation does not proceed very efficiently. In addition to oxidation of cumene and dehydrogenation of isopropyl alcohol, direct atmospheric oxidation of propylene via a 2-stage system with Pd(II) salts, Cu(II)Cl₂ and acetic acid (Wacker-Hoechst process) is also of importance industrially. However, the disadvantage of this process lies in the use of a mixture of metal ions as the catalyst, as a result of which the separation and recovery of the noble metal palladium is made very difficult. Furthermore, carrying out the reaction under strongly acid conditions necessitates the use of expensive corrosion-resistant reactors. Another disadvantage of the Wacker-Hoechst process lies in the possible entrainment of residues of acid when separating off the organic product, necessitating additional purification steps.

BE 828603 discloses that the oxidation of propylene in the liquid phase can be shifted in the direction of a vinylic oxidation to acetone if other metal additives, for example heteropolyacids of molybdenum, such as, for example, PMo₄V₈O₄₀ or TeMo₃V₃O₂₄, are added to the palladium catalyst. However, the experiments described in this document were carried out at a pH of 1.0 and therefore require an acid-resistant reactor.

TROVOG B., MARES F. and DIAMOND S. (J. Am. Chem. Soc. 102 (1980): 6618) describe a process for the oxidation of propylene with molecular oxygen to give acetone in diglyme as the solvent, in which cobalt-nitro complexes are employed as co-catalysts, together with Pd precursors. The disadvantage here also lies in the complicated separating off and recovery of the noble metal palladium.

### BRIEF SUMMARY OF THE PRESENT INVENTION

Generally, the object according to the invention is to overcome the disadvantages resulting from the prior art.

The object according to the invention furthermore comprises providing a process in which unsaturated hydrocarbons can be subjected to selective allylic or vinylic oxidation by simple variation of the ligand.

Another object according to the invention comprised providing a process for the oxidation of unsaturated hydrocarbons, preferably propylene, which converts propylene selectively into acrylic acid or acetone in a liquid phase under moderate conditions.

The invention is furthermore based on the object of providing a process for the oxidation of propylene to acrylic acid in a liquid phase, wherein the liquid phase containing acrylic acid can subsequently be employed for the preparation of polymers based on acrylic acid, without prior purification. By using the liquid phase containing acrylic acid in the preparation of polymers, cost- and time-consuming concentration steps on the acrylic acid, such as are hitherto customary, can be avoided. This concentration of the acrylic acid is already uneconomical because in the preparation of polymers by solution polymerization or inverse emulsion polymerization, the acrylic acid must in any case first be dissolved again in water.

### DETAILED DESCRIPTION OF THE INVENTION

The above objects are achieved by a process for the oxidation of unsaturated hydrocarbons, wherein an unsaturated hydrocarbon, an oxygen-containing oxidizing agent, a palladium complex as the catalyst containing one, preferably two, ligands of the formula (I) 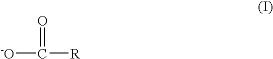
wherein R is a saturated, halogenated alkyl radical having from about 1 to about 20 carbon atoms, preferably having up to about 10 carbon atoms, and particularly preferably having up to about 5 carbon atoms,
and optionally auxiliary substances are brought into contact with one another in a liquid phase based on
(α1) from about 10 to about 100 vol. %, preferably from about 40 to about 90 vol. % and particularly preferably from about 50 to about 75 vol. % of a protic polar solvent and
(α2) from 0 to about 90 vol. %, preferably from about 10 to about 60 vol. % and particularly preferably from about 25 to about 50 vol. % of an aprotic polar solvent, the sum of components (α1) and (α2) being about 100 vol. %,
   at a temperature in a range from about 30 to about 300° C., preferably in a range from about 45 to about 200° C. and particularly preferably in a range from about 60 to about 120° C., under a pressure in a range from about 1 to about 200 bar, preferably in a range from about 5 to about 150 bar and particularly preferably in a range from about 10 to about 80 bar, such that, preferably, as a result of which a liquid phase containing oxygen-containing hydrocarbons is obtained.

In a particular embodiment of the process according to the invention, a mixture based on
(α1) a protic polar solvent and
(α2) an aprotic polar solvent, the weight ratio of the protic to the aprotic solvent being in a range from about 100,000:1 to about 1:10, particularly preferably in a range from about 1,000:1 to about 1:10 and more preferably in a range from about 10:1 to about 1:10,
   is employed as the liquid phase.

Unsaturated hydrocarbons which are employed in the process according to the invention are preferably olefins having from about 2 to about 60 carbon atoms, which can be unbranched or branched, mono- or polyunsaturated and optionally substituted, and can be described by the formula (II) 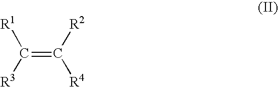
wherein R¹, R², R³ and R⁴ independently of one another can be hydrogen, an optionally branched C₁-C₈-alkyl, a straight-chain or branched C₁-C₈-alkenyl, a phenyl radical or naphthyl radical, or wherein two of the radicals R¹ to R⁴ together can form an alkylene chain —(CH₂)ₘ—, wherein m=about 3 to about 10, preferably about 4 to about 9 and particularly preferably about 5 to about 8, with the condition that at least one of the radicals R¹ to R⁴ is either a hydrogen or a methyl group. Particularly preferred unsaturated hydrocarbons which are employed in the process according to the invention are chosen from the group consisting of propylene, isobutene, n-hexene, hexadienes, in particular 1,5-hexadiene, n-octene, decene, dodecene, 1,9-decadiene, 2-methyl-1-butene, 2,3-dimethyl-2-butene, 2-methyl-1-hexene, 1,3-butadiene, 3-methyl-1,3-butadiene, octadecene, 2-ethyl-1-butene, styrene, cyclopentene, cyclohexene, 1-methyl-1-cyclohexene, cycloheptene, cyclooctene, cyclooctadiene, cyclododecene, cyclododecatriene, cyclohexadecadiene or limonene, propylene being particularly preferred.

Oxygen-containing oxidizing agents which are employed in the process according to the invention are preferably oxidizing agents which are capable of transferring at least one oxygen atom to the hydrocarbon under the reaction conditions described. Preferred oxygen-containing oxidizing agents are molecular oxygen (O₂), hydrogen peroxide (H₂O₂) and dinitrogen monoxide (N₂O), O₂ being particularly preferred. If O₂ is employed as the oxidizing agent, it is furthermore preferable for the oxygen to be employed as a mixture with one or more inert gases, such as nitrogen, argon or CO₂, or in the form of air.

The halogenated radical R in the ligand of the palladium compound of the formula (I) is preferably a fluorinated branched or unbranched alkyl radical, particularly preferably a branched or unbranched perfluoroalkyl radical having from about 1 to 10 carbon atoms, for example pentafluoroethyl or trifluoromethyl. A radical R which is particularly preferred in this connection is the trifluoromethyl group (—CF₃).

The palladium complexes are prepared in the manner known to the expert, for example by reaction of a salt of an anion of the formula (I) with a palladium salt, preferably with PdCl₂, in aqueous solution. Pd(CF₃)₂ is commercially obtainable, for example from ACROS, Belgium.

In a preferred embodiment of the process according to the invention, no further transition metals of sub-group VIII apart from palladium, and preferably no transition metals, are employed.

In another preferred embodiment of the process according to the invention, the palladium complex contains, in addition to the ligand of the formula (I), an organic ligand (X∩Y) which contains at least two atoms X and Y of main group III, V or VI of the periodic table, wherein this ligand can be coordinated to palladium via at least one of the two atoms X and Y an wherein at least one of these atoms is a constituent of a heterocyclic, aromatic ring system. The two atoms X and Y here can be identical or different. The selectivity of the oxidation of unsaturated hydrocarbons is shifted to the formation of ketones by the use of this ligand.

In a preferred embodiment of the organic ligand (X∩Y), this can be coordinated to palladium as a bidentate ligand via the two atoms X and Y.

A particularly preferred ligand (X∩Y) which can be coordinated to the palladium in addition to the ligand of the formula (I) is an organic ligand which contains from about 5 to about 50, preferably from about 10 to about 26 carbon atoms and at least two atoms from the following main groups or combinations of main groups of the periodic table: III and III, V and V, VI and VI, III and V, III and VI, V and VI, the combination V and V being particularly preferred. Each of the main groups or combinations of main groups of the periodic table here represents a preferred embodiment of a ligand (X∩Y) bonded to the palladium complex.

It is furthermore preferable for the ligand (X∩Y), which can be coordinated to the palladium in addition to the ligand of the formula (I), to have at least the following structural element (III) with conjugated double bonds: 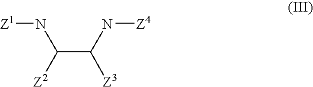
wherein at least two of the radicals Z¹ to Z⁴, preferably Z¹ and Z², Z¹ and Z³, Z¹ and Z⁴, Z² and Z³, Z² and Z⁴ and Z³ and Z⁴, where Z¹ and Z², Z² and Z³ and Z³ and Z⁴ are particularly preferred, are bonded to one another to form an aromatic ring system, preferably having from about 8 to about 30, particularly preferably from about 8 to about 26 carbon atoms, and preferably from about 2 to about 8, particularly preferably from about 2 to about 5 rings.

Ligands which are particularly preferred in this connection are chosen from the group consisting of 2,2′-bipyridyl (1), o-phenanthroline (2), bathophen-sulfonate (3), bathocuproin (4), 2,2′-biquinoyl (5), 3,6-di-(2-pyridyl)-1,2,4,5-tetrazine (6), 2,2′-bipyrimidine (7) and 2,3-di-(2-pyridyl)-pyrazine (8), where 2,2′-bipyridyl (1) and bathophen-sulfonate (3) are particularly preferred. In addition to this, it is preferable for the SO₃⁻ groups in the compound (3) to be in the para-position.

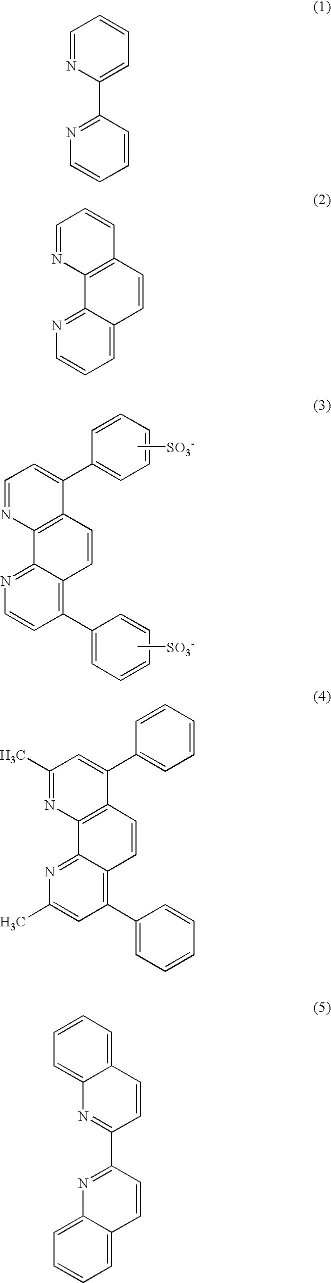

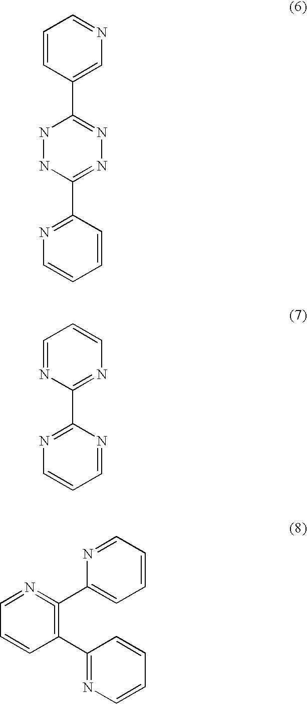

If a palladium complex containing ligands of the formula (I) is employed as the catalyst, salts, co-catalysts, further co-ligands or promoters can be employed as auxiliary substances in the process according to the invention. This applies in particular if a palladium complex containing ligands of the formula (I) but no organic ligands (X∩Y) is employed as the catalyst. Salts which are preferably employed here are KClO₄, NaCl, Cs₂CO₃, Na(CH₃COO) or Na(CF₃COO). Preferred co-catalysts are metal additives, for example Cu(BF₄)₂, Ag(CF₃COO), Co(salen), SnSO₄, Fe(acac)₃, Mo(acac)₃, MoO₂(acac)₂, K₂Cr₂O₇, Mn(CH₃COO)₃, Co(CH₃COO)₂, or Ni(CF₃COO)₂. Preferred co-ligands are 18-crown-6,15-crown-5, hexafluoroacetylacetonate, trifluoroacetylacetonate or acetylacetonate. Promoters which are preferably employed are methyl iodide or free radical initiators, such as N-hydroxy-phthalimide (NHPI).

The co-ligands and palladium are preferably employed in the process according to the invention in a molar ratio of co-ligand:palladium in a range from about 20:1 to about 4:1, particularly preferably in a molar ratio in a range from about 12:1 to about 8:1. The salts are preferably employed in the process according to the invention in a concentration in a range from about 0.1 to about 10 mmol/l, particularly preferably in a range from about 0.5 to about 5 mmol/l. The promoters are preferably present in the process according to the invention in a concentration in a range from about 0.1 to about 10 mmol/l, particularly preferably in a range from about 0.5 to about 1 mmol/l. The co-catalysts are preferably employed in the process according to the invention in an amount such that the molar ratio between the metal of the co-catalyst and the palladium is in a range from about 0.5:1 to about 2:1, preferably in a range from about 0.9:1 to about 1.1:1.

If a palladium complex containing ligands of the formula (I) but no further organic ligands (X∩Y) is employed as the catalyst, in a preferred embodiment of the process according to the invention acetic acid or a salt of acetic acid is employed as an auxiliary substance. The sodium salt and the potassium salt and mixtures thereof are preferred as the salt of acetic acid, the sodium salt being particularly preferred. It is furthermore preferable in this connection for the acetic acid or the salt of acetic acid to be employed in an amount such that the CH₃COO— group is present in the liquid phase in protonated or non-protonated form in a concentration in a range from about 0.001 to about 100 mmol/l, preferably in a range from about 0.01 to about 50 mmol/l and particularly preferably in a range from about 0.1 to about 10 mmol/l.

Water, methanol and ethanol, acetic acid, trifluoroacetic acid and mixtures of at lest two of these are preferably employed as the protic polar solvent in the process according to the invention, water and mixtures of water and trifluoroacetic acid in a weight ratio of water/trifluoroacetic acid in a range from about 10:1 to about 1:1, preferably from about 5:1 to about 3:1, being particularly preferred.

Aprotic polar solvents which are preferably employed are polyethylene glycol dialkyl ethers, polyethylene glycol divinyl ethers or polyethylene glycol vinyl alkyl ethers. Diethylene glycol dimethyl ether, triethylene glycol dimethyl ether, diethylene glycol methyl vinyl ether, triethylene glycol methyl vinyl ether, diethylene glycol divinyl ether, triethylene glycol divinyl ether, triethylene glycol diethyl ether, diethylene glycol diethyl ether and dimethylpropyleneurea (DMPU) are preferred among these, diethylene glycol dimethyl ether (diglyme) being particularly preferred.

In a particularly preferred embodiment of the process according to the invention, a mixture of water and diglyme is employed as the liquid phase. In this connection it is preferable for the water and diglyme to be employed in the liquid phase in a weight ratio of water:diglyme in a range from about 100,000:1 to about 1:10, particularly preferably in a range from about 1,000:1 to about 1:10 and more preferably in a range from about 10:1 to about 1:10.

The pH of the liquid phase is preferably in a range from 0 to about 12, particularly preferably in a range from about 1 to about 11 and more preferably in a range from about 2 to about 10.

The unsaturated hydrocarbon, the oxygen-containing oxidizing agent, the palladium complex and optionally the auxiliary substances are preferably brought into contact by first dissolving the catalyst, optionally with the auxiliary substances, in the liquid phase. If the catalyst contains the organic ligand (X∩Y) in addition to a ligand of the formula (I), before being brought into contact with the unsaturated hydrocarbon and the oxygen-containing oxidizing agent the palladium complex is prepared by reaction of a palladium compound of the formula (III) 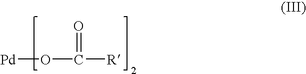
wherein the radical R′ has the same meaning as the radical R described above, with the organic ligand (X∩Y) in a molar ratio in a range from about 1:5 to about 5:1, preferably in a range from about 1:2 to about 2:1, and particularly preferably in a molar ratio of about 1:1. The reaction is preferably carried out at a temperature in a range from about 20 to about 80° C. under a pressure in a range from about 1 to about 20 bar. In this connection, it is furthermore preferable for the preparation of the palladium complexes to be carried out in situ. It is also possible for this palladium complex to be prepared in a separate batch by reaction of the palladium compound with the organic ligand in the liquid phase and for the palladium complex prepared in this manner then to be transferred into the reaction vessel in which the oxidation of the unsaturated hydrocarbon takes place. The liquid phase in which the palladium complex is prepared preferably corresponds here in its chemical composition to the liquid phase in which the oxidation of the unsaturated hydrocarbon takes place. In this connection it is furthermore preferable for the abovementioned palladium compound to be reacted with a mixture comprising at least two structurally different organic ligands (X∩Y) for the preparation of a palladium complex.

In another preferred embodiment of the process according to the invention, the palladium complex is immobilized on a support and the support with the immobilized palladium complex is then introduced into the liquid phase. Supports which are preferably employed are aluminium hydroxide, silica gel, aluminium oxide, aluminium silicate, pumice, zeolites, tin oxides, preferably SnO₂, titanium oxides, preferably TiO₂, or active charcoal. The palladium complex is preferably immobilized by immersing the support in a solution containing the palladium complex or by impregnating the support with a solution containing the palladium complex at a temperature in a range from about 20 to about 150° C. under a pressure in a range from about 5 to about 100 bar. It is furthermore possible to bond the catalyst chemically to a support via suitable functional groups on one of the ligands.

In a preferred embodiment of the process according to the invention, the palladium complex is present in the liquid phase in a concentration in a range from about 0.001 to about 100 mmol/l, preferably in a range from about 0.01 to about 10 mmol/l and particularly preferably in a range from about 0.1 to about 1 mmol/l.

If the oxygen-containing oxidizing agent is H₂O₂, this is added to the liquid phase together with the catalyst or the catalyst immobilized on a support. If the oxygen-containing oxidizing agent is gaseous, this is brought into contact, together with the unsaturated hydrocarbon under pressure, with the liquid phase containing the palladium complex and optionally the auxiliary substances, preferably with vigorous stirring of the liquid phase, and the mixture is heated to the appropriate reaction temperature. On a large industrial scale, the liquid phase can be brought into contact with the gaseous oxygen-containing oxidizing agent, for example, in a trickle bed with a bubble phase. In all cases, the liquid phase must be brought into contact with the oxygen-containing oxidizing agent in a manner such that the unsaturated hydrocarbon is oxidized by the oxygen-containing oxidizing agent to form an oxygen-containing hydrocarbon.

In a preferred embodiment of the process according to the invention, the palladium complex is first activated by reduction, preferably to increase the selectivity of the oxidation reaction, before it catalyses the oxidation of the unsaturated hydrocarbon. In a preferred embodiment the reduction of the palladium complex is carried out by hydrogen gas. For this, the hydrogen gas is brought into contact, before the oxidizing agent and preferably under a pressure in a range from about 1 to about 20 bar at a temperature in a range from about 20 to about 80° C. in a pressure vessel and while stirring, with the palladium complex, which is preferably dissolved or dispersed in the aqueous phase.

In another preferred embodiment, the reduction of the palladium complex is effected by the unsaturated hydrocarbon. For this purpose, this is employed with the oxidizing agent in the process according to the invention in a molar ratio of unsaturated hydrocarbon/oxidizing agent of at least about 1, preferably at least about 2 and particularly preferably at least about 3. Reduction of the Pd catalyst with the unsaturated hydrocarbon before the start of the reaction minimizes the vinylic oxidation to the ketone already at the start of the reaction.

The period during which the unsaturated hydrocarbon, the oxygen-containing oxidizing agent and the palladium complex are brought into contact under the conditions described above depends on the individual process parameters, in particular on the amounts of e-ducts employed. However, the reaction is carried out under the stated conditions at least until a sufficient amount of the unsaturated hydrocarbon employed, preferably at least about 10%, particularly preferably at least about 20% and more preferably at least about 70% is converted, that is to say has been oxidized by the oxidizing agent, the extent of the conversion being determined by the test method described herein. In a preferred embodiment of the process according to the invention, the individual components are brought into contact under the process conditions for at least about one hour, particularly preferably for at least about 2 hours. The reaction is preferably ended by ending the contact of the unsaturated hydrocarbon with the palladium compound in the liquid phase under the abovementioned pressure, preferably by pressure compensation between the reaction vessel and the surrounding atmosphere.

If a palladium complex which contains ligands of the formula (I) but no further organic ligands (X∩Y) is employed as the catalyst in the process according to the invention, the corresponding α,β-unsaturated carboxylic acid is obtained as the reaction product to an increased extent, preferably with a selectivity, determined in accordance with the method described herein, in a range from about 10 to about 99%, particularly preferably in a range from about 20 to about 75% and more preferably in a range from about 29 to about 53%, provided that at least one of the radicals R¹ to R⁴ corresponds to a methyl group. In the case of propylene, if such a palladium complex is used acrylic acid is accordingly obtained with a high selectivity, preferably in a range from about 10 to about 99%, particularly preferably in a range from about 20 to about 75% and more preferably in a range from about 29 to about 53%. In this connection it is furthermore preferable for the value of the specific catalyst output (═SCO value), determined in accordance with the methods described herein, for the synthesis of the α,β-unsaturated carboxylic acid from the corresponding unsaturated hydrocarbon, preferably for the synthesis of acrylic acid from propylene, to be at least about 1 g/g_{Pd}/h, particularly preferably at least about 100 g/g_{Pd}/h and more preferably at least about 1,000 g/g_{Pd}/h, where an SCO value of about 10,000 g/g_{Pd}/h is preferably not exceeded.

If a palladium complex which contains both a ligand of the formula (I) and the organic ligand (X∩Y) as ligands is employed as the catalyst in the process according to the invention, the corresponding carbonyl compound is obtained as the reaction product to an increased extent, preferably with a selectivity, determined in accordance with the method described herein, in a range from about 60 to about 90%, preferably in a range from about 65 to about 85% and particularly preferably in a range from about 70 to about 80%, provided that at least one of the radicals R¹ to R⁴ corresponds to a hydrogen atom. In the case of propylene, if such a palladium complex is used acetone is accordingly obtained with a high selectivity, preferably in a range from about 60 to about 90%, preferably in a range from about 65 to about 85% and particularly preferably in a range from about 70 to about 80%. In this connection it is furthermore preferable for the SCO value, determined in accordance with the methods described herein, for the synthesis of the carbonyl compound from the corresponding unsaturated hydrocarbon, preferably for the synthesis of acetone from propylene, to be at least about 1 g/g_{Pd}/h, particularly preferably at least about 100 g/g_{Pd}/h and more preferably at least about 1,000 g/g_{Pd}/h, where an SCO value of about 10,000 g/g_{Pd}/h is preferably not exceeded.

The invention furthermore relates to the oxidized hydrocarbons obtainable by the process according to the invention.

The invention also relates to the liquid phase obtainable by the process according to the invention containing oxidized hydrocarbons.

The invention also relates to the use of the oxidized hydrocarbons obtainable by the process according to the invention in chemical products, preferably in fibres, films and water-absorbent polymer structures, which are preferably employed in the production of hygiene articles, such as diapers and other incontinence products, as well as sanitary towels.

The invention moreover relates to chemical products comprising the oxidized hydrocarbons obtainable by the process according to the invention, the abovementioned chemical products being preferred as the chemical products.

The invention moreover relates to the reduced palladium complexes described above and the use thereof for the oxidation of unsaturated hydrocarbons in the liquid phase.

The invention also relates to the use of acetic acid or of a salt of acetic acid in the process according to the invention, wherein a palladium complex containing a ligand of the formula (I) but no further organic ligands (X∩Y) is employed as the catalyst,
(δ1) to increase the SCO value of the palladium complex in the oxidation of unsaturated hydrocarbons, preferably in the oxidation of propylene, or
(δ2) to increase the selectivity of the oxidation of unsaturated hydrocarbons, preferably of propylene.

Preferred embodiments of the use according to the invention of acetic acid or of the salt of acetic acid result from the following uses or combinations of uses: δ1, δ2, δ1δ2.

Preferred salts of acetic acid and ligands of the formula (I) are those compounds which have already been described in connection with the process according to the invention for the oxidation of unsaturated hydrocarbons. The palladium complex is preferably prepared in the manner such as has been described in connection with the process according to the invention for the oxidation of unsaturated hydrocarbons.

Preferably, increasing the SCO value (δ1) is understood as increasing the SCO value compared with the SCO value of the oxidation of an unsaturated hydrocarbon with the same palladium complex but in the absence of acetic acid or the salt of acetic acid. In this connection it is furthermore preferable for the increase in the SCO value to be at least about 20%, preferably at least about 30%, in each case based on the SCO value in the absence of acetic acid or the salt of acetic acid.

Increasing the selectivity (δ2) is preferably understood as increasing the selectivity compared with the selectivity of the oxidation of an unsaturated hydrocarbon with the same palladium complex but in the absence of acetic acid or the salt of acetic acid, with the same conversion, that is to say at the same conversion of the unsaturated hydrocarbon. In this connection it is furthermore preferable for the increase in the selectivity to be at least about 50%, preferably at least about 100%, in each case based on the selectivity in the absence of acetic acid or the salt of acetic acid.

The invention also relates to a process for the preparation of water-soluble or water-absorbent polymers, wherein, in a liquid phase obtainable by the process according to the invention for the oxidation of unsaturated hydrocarbons in which a palladium complex containing ligands of the formula (I) but preferably no further organic ligands (X∩Y) is employed as the catalyst, the α,β-unsaturated carboxylic acid contained as the oxygen-containing hydrocarbon in the liquid phase is polymerized and the water-soluble or water-absorbent polymer obtained in this way is then optionally dried and comminuted.

In a preferred embodiment of the process according to the invention for the preparation of water-soluble or water-absorbent polymers, that liquid phase which is obtainable by the process according to the invention for the oxidation of unsaturated hydrocarbons in which water or a mixture of water and diglyme, preferably in a weight ratio of water: diglyme in a range from about 10,000:1 to about 100:1, is employed as the liquid phase and propylene is employed as the unsaturated hydrocarbon is employed as the liquid phase. The liquid phase is accordingly preferably an aqueous acrylic acid solution.

Preferred ligands of the formula (I) are those compounds which have already been described in connection with the process according to the invention for the oxidation of unsaturated hydrocarbons. The palladium complex containing ligands of the formula (I) is preferably prepared in a manner such as has been described in connection with the process according to the invention for the oxidation of unsaturated hydrocarbons.

It is furthermore preferable in the process according to the invention for the preparation of water-soluble or water-absorbent polymers for the α,β-unsaturated carboxylic acid contained in the liquid phase to be copolymerized with further monomers which can be copolymerized with the α,β-unsaturated carboxylic acid. These monomers are preferably compounds chosen from the group consisting of (β1) ethylenically unsaturated monomers containing acid groups or salts thereof or polymerized, ethylenically unsaturated monomers containing a protonated or quaternized nitrogen, or mixtures thereof, (β2) ethylenically unsaturated monomers which can be copolymerized with (β1), and (β3) crosslinking agents.

The ethylenically unsaturated monomers (β1) containing acid groups and the α,β-unsaturated carboxylic acid contained in the liquid phase obtainable by the process according to the invention for the oxidation of unsaturated hydrocarbons can be partly or completely, preferably partly, neutralised. Preferably, the monoethylenically unsaturated monomers (β1) containing acid groups and the α,β-unsaturated carboxylic acid are neutralized to the extent of at least about 25 mol %, particularly preferably to the extent of at least about 50 mol % and more preferably to the extent of about 50- about 90 mol %. The neutralization of the monomers (β1) and of the α,β-unsaturated carboxylic acid can be carried out before and also after the polymerization. Furthermore, the neutralization can be carried out with alkali metal hydroxides, alkaline earth metal hydroxides, ammonia and carbonates and bicarbonates. In addition, any further base which forms a water-soluble salt with the acid is conceivable. Mixed neutralization with various bases is also conceivable. Neutralization with ammonia or with alkali metal hydroxides is preferred, particularly preferably with sodium hydroxide or with ammonia.

Preferred monoethylenically unsaturated monomers (β1) containing acid groups which can be employed alongside the α,β-unsaturated carboxylic acid contained in the liquid phase obtainable by the process according to the invention for the oxidation of unsaturated hydrocarbons are acrylic acid, methacrylic acid, ethacrylic acid, α-chloroacrylic acid, α-cyanoacrylic acid, β-methylacrylic acid (crotonic acid), α-phenylacrylic acid, β-acryloxypropionic acid, sorbic acid, α-chlorosorbic acid, 2′-methylisocrotonic acid, cinnamic acid, p-chlorocinnamic acid, β-stearyl acid, itaconic acid, citraconic acid, mesaconic acid, glutaconic acid, aconitic acid, maleic acid, fumaric acid, tricarboxyethylene and maleic anhydride, where acrylic acid and methacrylic acid are particularly preferred.

In addition to these monomers containing carboxylate groups, ethylenically unsaturated sulfonic acid monomers or ethylenically unsaturated phosphonic acid monomers are furthermore preferred as monoethylenically unsaturated monomers (β1) containing acid groups.

Preferred ethylenically unsaturated sulfonic acid monomers are allylsulfonic acid or aliphatic or aromatic vinylsulfonic acids or acrylic or methacrylic sulfonic acids. Preferred aliphatic or aromatic vinylsulfonic acids are vinylsulfonic acid, 4-vinylbenzenesulfonic acid, vinyltoluenesulfonic acid and styrenesulfonic acid. Preferred acrylo- or methacrylosulfonic acids are sulfoethyl (meth)acrylate, sulfopropyl (meth)acrylate and 2-hydroxy-3-methacryloxypropylsulfonic acid. 2-Acrylamido-2-methylpropanesulfonic acid is the preferred (meth)acrylamidoalkylsulfonic acid.

Ethylenically unsaturated phosphonic acid monomers, such as vinylphosphonic acid, allylphosphonic acid, vinylbenzylphosphonic acid, (meth)acrylamidoalkylphosphonic acids, acrylamidoalkyldiphosphonic acids, phosphonomethylated vinylamines and (meth)acrylophosphonic acid derivatives, are furthermore preferred.

Preferred ethylenically unsaturated monomers (β1) containing a protonated nitrogen are, preferably, dialkylaminoalkyl (meth)acrylates in protonated form, for example dimethylaminoethyl (meth)acrylate hydrochloride or dimethylaminoethyl (meth)acrylate hydrosulfate, and dialkylaminoalkyl-(meth)acrylamides in protonated form, for example dimethylaminoethyl(meth)acrylamide hydrochloride, dimethylaminopropyl(meth)acrylamide hydrochloride, dimethylaminopropyl(meth)acrylamide hydrosulfate or dimethylaminoethyl(meth)acrylamide hydrosulfate.

Preferred ethylenically unsaturated monomers (β1) containing a quaternized nitrogen are dialkylammoniumalkyl (meth)acrylates in quaternized form, for example trimethylammoniumethyl (meth)acrylate methosulfate or dimethylethylammoniumethyl (meth)acrylate ethosulfate, and (meth)acrylamidoalkyldialkylamines in quaternized form, for example (meth)acrylamidopropyltrimethylammonium chloride, trimethylammoniumethyl (meth)acrylate chloride or (meth)acrylamidopropyltrimethylammonium sulfate.

Preferred monoethylenically unsaturated monomers (β2) which can be copolymerized with (β1) are acrylamides and methacrylamides.

Possible (meth)acrylamides are, in addition to acrylamide and methacrylamide, alkyl-substituted (meth)acrylamides or aminoalkyl-substituted derivatives of (meth)acrylamide, such as N-methylol(meth)acrylamide, N,N-dimethylamino(meth)acrylamide, dimethyl(meth)acrylamide or diethyl(meth)acrylamide. Possible vinylamides are, for example, N-vinylamides, N-vinylformamides, N-vinylacetamides, N-vinyl-N-methylacetamides, N-vinyl-N-methylformamides, vinylpyrrolidone. Among these monomers, acrylamide is particularly preferred.

Water-dispersible monomers are furthermore preferred as monoethylenically unsaturated monomers (β2) which can be copolymerized with (β1). Preferred water-dispersible monomers are acrylic acid esters and methacrylic acid esters, such as methyl (meth)acrylate, ethyl (meth)acrylate, propyl (methyl)acrylate or butyl (meth)acrylate, as well as vinyl acetate, styrene and isobutylene.

Crosslinking agents (β3) which are preferred according to the invention are compounds which contain at least two ethylenically unsaturated groups within a molecule (crosslinking agent class I), compounds which contain at least two functional groups which can react with functional groups of monomers (β1) or (β2) in a condensation reaction (=condensation-crosslinking agents), in an addition reaction or in a ring-opening reaction (crosslinking agent class II), compounds which contain at least one ethylenically unsaturated group and at least one functional group which can react with functional groups of monomers (β1) or (β2) in a condensation reaction, in an addition reaction or in a ring-opening reaction (crosslinking agent class III), or polyvalent metal cations (crosslinking agent class IV). A crosslinking of the polymers by free-radical polymerization of the ethylenically unsaturated groups of the crosslinking agent molecule with the monoethylenically unsaturated monomers (β1) or (β2) is achieved here by the compounds of crosslinking agent class I, while in the case of the compounds of crosslinking agent class II and the polyvalent metal cations of crosslinking agent class IV crosslinking of the polymers is achieved by a condensation reaction of the functional groups (crosslinking agent class II) or by electrostatic interaction of the polyvalent metal cation (crosslinking agent class IV) with the functional groups of monomers (β1) or (β2). In the case of the compounds of crosslinking agent class III crosslinking of the polymer accordingly takes place both by free-radical polymerization of the ethylenically unsaturated group and by a condensation reaction between the functional group of the crosslinking agent and the functional groups of monomers (β1) or (β2).

Preferred compounds of crosslinking agent class I are poly(meth)acrylic acid esters, which are obtained, for example, by reaction of a polyol, such as, for example, ethylene glycol, propylene glycol, trimethylolpropane, 1,6-hexanediol, glycerol, pentaerythritol, polyethylene glycol or polypropylene glycol, an amino alcohol, a polyalkylene-polyamine, such as, for example, diethylenetriamine or triethylenetetramine, or an alkoxylated polyol with acrylic acid or methacrylic acid. Preferred compounds of crosslinking agent class I are furthermore polyvinyl compounds, poly(meth)allyl compounds, (meth)acrylic acid esters of a monovinyl compound or (meth)acrylic acid esters of a mono(meth)allyl compound, preferably of the mono(meth)allyl compounds of a polyol or of an amino alcohol.

Examples of compounds of crosslinking agent class I which may be mentioned are alkenyl di(meth)acrylates, for example ethylene glycol di(meth)acrylate, 1,3-propylene glycol di(meth)acrylate, 1,4-butylene glycol di(meth)acrylate, 1,3-butylene glycol di(meth)acrylate, 1,6-hexanediol di(meth)acrylate, 1,10-decanediol di(meth)acrylate, 1,12-dodecanediol di(meth)acrylate, 1,18-octadecanediol di(meth)acrylate, cyclopentanediol di(meth)acrylate, neopentylglycol di(meth)acrylate, methylene di(meth)acrylate or pentaerythritol di(meth)acrylate, alkenyldi(meth)acrylamides, for example N-methyldi(meth)acrylamide, N,N′-3-methylbutylidenebis(meth)acrylamide, N,N′-(1,2-dihydroxyethylene)bis(meth)acrylamide, N,N′-hexamethylenebis(meth)acrylamide or N,N′-methylenebis(meth)acrylamide, polyalkoxy-di(meth)acrylates, for example diethylene glycol di(meth)acrylate, triethylene glycol di(meth)acrylate, tetraethylene glycol di(meth)acrylate, dipropylene glycol di(meth)acrylate, tripropylene glycol di(meth)acrylate or tetrapropylene glycol di(meth)acrylate, bisphenol A di(meth)acrylate, ethoxylated bisphenol A di(meth)acrylate, benzylidene di(meth)acrylate, 1,3-di(meth)acryloyloxy-propan-2-ol, hydroquinone di(meth)acrylate, di(meth)acrylate esters of trimethylolpropane oxyalkylated, preferably ethoxylated, preferably with from about 1 to about 30 mol of alkylene oxide per hydroxyl group, thioethylene glycol di(meth)acrylate, thiopropylene glycol di(meth)acrylate, thiopolyethylene glycol di(meth)acrylate, thiopolypropylene glycol di(meth)acrylate, divinyl ethers, for example 1,4-butanediol-divinyl ether, divinyl esters, for example divinyl adipate, alkanedienes, for example butadiene or 1,6-hexadiene, divinylbenzene, di(meth)allyl compounds, for example di(meth)allyl phthalate or di(meth)allyl succinate, homo- and copolymers of di(meth)allyldimethylammonium chloride and homo- and copolymers of diethyl (meth)allylaminomethyl(meth)acrylate-ammonium chloride, vinyl(meth)acrylyl compounds, for example vinyl (meth)acrylate, (meth)allyl-(meth)acrylyl compounds, for example (meth)allyl (meth)acrylate, (meth)allyl (meth)acrylate ethoxylated with from about 1 to about 30 mol of ethylene oxide per hydroxyl group, di(meth)allyl esters of polycarboxylic acids, for example di(meth)allyl maleate, di(meth)allyl fumarate, di(meth)allyl succinate or di(meth)allyl terephthalate, compounds with 3 or more ethylenically unsaturated groups which can be polymerized by free radicals, such as, for example, glycerol tri(meth)acrylate, (meth)acrylate esters of glycerol oxyethylated with preferably from about 1 to about 30 mol of ethylene oxide per hydroxyl group, trimethylolpropane tri(meth)acrylate, tri(meth)acrylate esters of trimethylolpropane oxyalkylated, preferably ethoxylated, preferably with from about 1 to about 30 ml of alkylene oxide per hydroxyl group, trimethylacrylamide, (meth)allylidene di(meth)acrylate, 3-allyloxy-1,2-propanediol di(meth)acrylate, tri(meth)allyl cyanurate, tri(meth)allyl isocyanurate, pentaerythritol tetra(meth)acrylate, pentaerythritol tri(meth)acrylate, (meth)acrylic acid esters of pentaerythritol oxyethylated with preferably 1 to 30 mol of ethylene oxide per hydroxyl group, tris(2-hydroxyethyl) isocyanurate tri(meth)acrylate, trivinyl trimellitate, tri(meth)allylamine, di(meth)allylalkylamines, for example di(meth)allylmethylamine, tri(meth)allyl phosphate, tetra(meth)allylethylenediamine, poly(meth)allyl esters, tetra(meth)allyloxyethane or tetra(meth)allylammonium halides.

Compounds which contain at least two functional groups which can react with the functional groups of monomers (β1) or (β2), preferably with acid groups of monomers (β1), in a condensation reaction (=condensation-crosslinking agents), in an addition reaction or in a ring-opening reaction are preferred as the compound of crosslinking agent class II. These functional groups of compounds of crosslinking agent class II are preferably alcohol, amine, aldehyde, -glycidyl, isocyanate, carbonate or epichloro functions.

Examples which may be mentioned of the compound of crosslinking agent class II are polyols, for example ethylene glycol, polyethylene glycols, such as diethylene glycol, triethylene glycol and tetraethylene glycol, propylene glycol, polypropylene glycols, such as dipropylene glycol, tripropylene glycol or tetrapropylene glycol, 1,3-butanediol, 1,4-butanediol, 1,5-pentanediol, 2,4-pentanediol, 1,6-hexanediol, 2,5-hexanediol, glycerol, polyglycerol, trimethylolpropane, polyoxypropylene, oxyethylene/oxypropylene block copolymers, sorbitan fatty acid esters, polyoxyethylene sorbitan fatty acid esters, pentaerythritol, polyvinyl alcohol and sorbitol, amino alcohols, for example ethanolamine, diethanolamine, triethanolamine or propanolamine, polyamine compounds, for example ethylenediamine, diethylenetriamine, triethylenetetraamine, tetraethylenepentaamine or pentaethylenehexaamine, polyglycidyl ether compounds, such as ethylene glycol diglycidyl ether, polyethylene glycol diglycidyl ether, glycerol diglycidyl ether, glycerol polyglycidyl ether, pentaerythritol polyglycidyl ether, propylene glycol diglycidyl ether, polypropylene glycol diglycidyl ether, neopentylglycol diglycidyl ether, hexanediol glycidyl ether, trimethylolpropane polyglycidyl ether, sorbitol polyglycidyl ether, phthalic acid diglycidyl ester, adipic acid diglycidyl ether, 1,4-phenylene-bis(2-oxazoline), glycidol, polyisocyanates, preferably diisocyanates, such as 2,4-toluene diisocyanate and hexamethylene diisocyanate, polyaziridine compounds, such as 2,2-bishydroxymethylbutanol tris[3-(1-aziridinyl)propionate], 1,6-hexamethylenediethyleneurea and diphenylmethane-bis-4,4′-N,N′-diethyleneurea, halogenoepoxides, for example epichloro- and epibromohydrin and α-methylepichlorohydrin, alkylene carbonates, such as 1,3-dioxolan-2-one (ethylene carbonate), 4-methyl-1,3-dioxolan-2-one (propylene carbonate), 4,5-dimethyl-1,3-dioxolan-2-one, 4,4-dimethyl-1,3-dioxolan-2-one, 4-ethyl-1,3-dioxolan-2-one, 4-hydroxymethyl-1,3-dioxolan-2-one, 1,3-dioxan-2-one, 4-methyl-1,3-dioxan-2-one, 4,6-dimethyl-1,3-dioxan-2-one, 1,3-dioxolan-2-one, poly-1,3-dioxolan-2-one and polyquaternary amines, such as condensation products of dimethylamines and epichlorohydrin. Preferred compounds of crosslinking agent class II are furthermore polyoxazolines, such as 1,2-ethylenebisoxazoline, crosslinking agents with silane groups, such as γ-glycidoxypropyltrimethoxysilane and γ-aminopropyltrimethoxysilane, oxazolidinones, such as 2-oxazolidinone, bis- and poly-2-oxazolidinones and diglycol silicates.

Preferred compounds of class III are esters, containing hydroxyl or amino groups, of (meth)acrylic acid, such as, for example, 2-hydroxyethyl (meth)acrylate, and (meth)acrylamides containing hydroxyl or amino groups or mono(meth)allyl compounds of diols.

The polyvalent metal cations of crosslinking agent class IV are preferably derived from mono- or polyvalent cations, and the monovalent in particular from alkali metals, such as potassium, sodium and lithium, lithium being preferred. Preferred divalent cations are derived from zinc, beryllium and alkaline earth metals, such as magnesium, calcium and strontium, magnesium being preferred. Cations of higher valency which can furthermore be employed according to the invention are cations of aluminium, iron, chromium, manganese, titanium, zirconium and other transition metals, as well as double salts of such cations or mixtures of the salts mentioned. Aluminium salts and alums and various hydrates thereof, such as e.g. AlCl₃×6H₂O, NaAl(SO₄)₂×12H₂O, KAl(SO₄)₂×12H₂O or Al₂(SO₄)₃×14-18H₂O, are preferably employed.

Al₂(SO₄)₃ and its hydrates are particularly preferably used as crosslinking agents of crosslinking agent class IV.

Crosslinking agents of the following crosslinking agent classes and crosslinking agents of the following combinations of crosslinking agent classes are preferably employed in the process according to the invention for the preparation of water-soluble or water-absorbent polymers: I, II, III, IV, I II, I III, I IV, I II III, I II IV, I III IV, II III IV, II IV or III IV.

Further preferred embodiments of the process according to the invention are processes in which any desired one of the abovementioned crosslinking agents of crosslinking agent class I is employed as the crosslinking agent. Among these, water-soluble crosslinking agents are preferred. In this connection, N,N′-methylenebisacrylamide, polyethylene glycol di(meth)acrylates, triallylmethylammonium chloride, tetraallylammonium chloride and allyl-nonaethylene glycol acrylate prepared with about 9 mol of ethylene oxide per mol of acrylic acid are particularly preferred.

The abovementioned monomers and crosslinking agents are added, optionally with further adjuvants (β4), before the polymerization of the liquid phase which is obtainable by the process according to the invention for the oxidation of unsaturated hydrocarbons and contains the α,β-unsaturated carboxylic acid as oxygen-containing hydrocarbons. Preferred adjuvants (β4) in this connection are standardizing agents, odour-binding agents, surface-active agents or antioxidants. However, these adjuvants (β4) can also be added after the polymerization of the liquid phase or, after drying and comminution of the polymers, can be mixed with these. The water-soluble or water-absorbent polymer can be prepared by various polymerization procedures. In this connection there may be mentioned as examples solution polymerization, spray polymerization, inverse emulsion polymerization and inverse suspension polymerization. Solution polymerization is preferably carried out. A broad spectrum of possible variations in respect of reaction circumstances, such as temperatures, nature and amount of initiators and also the reaction solution, can be found from the prior art. Typical processes are described in the following patent specifications: U.S. Pat. No. 4,286,082, U.S. Pat. No. 4,179,367, U.S. Pat. No. 4,076,663, U.S. Pat. No. 4,587,308, U.S. Pat. No. 5,409,771, U.S. Pat. No. 5,610,220, U.S. Pat. No. 5,672,633, U.S. Pat. No. 5,712,316.

Polymerization initiators can be contained in the liquid phase in dissolved or dispersed form. Possible initiators are all the compounds known to the expert which dissociate into free radicals. These include, in particular, peroxides, hydroperoxides, hydrogen peroxide, persulfates, azo compounds and the so-called redox catalysts. The use of water-soluble catalysts is preferred. In some cases it is advantageous to use mixtures of various polymerization initiators. Among these mixtures, those of hydrogen peroxide and sodium peroxodisulfate or potassium peroxodisulfate, which can be employed in any conceivable ratio of amounts, are preferred. Suitable organic peroxides are, preferably, acetylacetone peroxide, methyl ethyl ketone peroxide, t-butyl hydroperoxide, cumene hydroperoxide, t-amyl perpivalate, t-butyl perpivalate, t-butyl pemeohexonate, t-butyl isobutyrate, t-butyl per-2-ethylhexenoate, t-butyl perisononanoate, t-butyl permaleate, t-butyl perbenzoate, t-butyl 3,5,5-tri-methylhexanoate and amyl perneodecanoate. Polymerization initiators which are furthermore preferred are: azo compounds, such as 2,2′-azobis-(2-amidinopropane) dihydrochloride, azo-bis-amidinopropane dihydrochloride, 2,2′-azobis-(N,N-dimethylene)isobutyramidine dihydrochloride, 2-(carbamoylazo)isobutyronitrile and 4,4′-azobis-(4-cyanovaleric acid). The compounds mentioned are employed in conventional amounts, preferably in a range from about 0.01 to about 5, preferably from about 0.1 to about 2 mol %, in each case based on the amount of monomers to be polymerized.

The redox catalysts contain as the oxidic component at least one of the above-mentioned per-compounds and as the reducing component, preferably, ascorbic acid, glucose, sorbose, mannose, ammonium or alkali metal hydrogen sulfite, sulfate, thiosulfate, hyposulfite or sulfide, metal salts, such as iron(II) ions or silver ions or sodium hydroxymethyl-sulfoxylate. Ascorbic acid or sodium pyrosulfite is preferably used as the reducing component of the redox catalyst. From about 1*10⁻⁵ to about 1 mol % of the reducing component of the redox catalyst and from about 1*10⁻⁵ to about 5 mol % of the oxidizing component of the redox catalyst are employed, based on the amount of monomers employed in the polymerization. Instead of the oxidizing component of the redox catalysts, or in addition to this, one or more, preferably water-soluble azo compounds can be used.

A redox system comprising hydrogen peroxide, sodium peroxodisulfate and ascorbic acid is preferably employed according to the invention. Generally, azo compounds are preferred according to the invention as initiators, azo-bis-amidinopropane dihydrochloride being particularly preferred. As a rule, the polymerization is initiated with the initiators in a temperature range from about 30 to about 90° C.

Another possibility for the preparation according to the invention of water-absorbent polymers is first to prepare non-crosslinked, in particular linear polymers, preferably by the free-radical route, from the α,β-unsaturated carboxylic acid and optionally the abovementioned monoethylenically unsaturated monomers (β1) or (β2) and then to react these with reagents (β3) having a crosslinking action, preferably those of classes II and IV. This variant is preferably employed if the water-absorbent polymers are first to be processed in shaping processes, for example to give fibres, films or other sheet-like structures, such as woven fabrics, knitted fabrics, spun fabrics or nonwovens, and are to be crosslinked in this form.

In another preferred embodiment of the process according to the invention for the preparation of water-soluble or water-absorbent polymers, in addition to the α,β-unsaturated carboxylic acid, preferably acrylic acid, and optionally to the further monomers (β1), (β2) and crosslinking agents (β3), water-soluble polymers (β5) are polymerized in. These water-soluble polymers (β5) are preferably partly or completely hydrolysed polyvinyl alcohol, polyvinylpyrrolidone, starch or starch derivatives, polyglycols or polyacrylic acid. The molecular weight of these polymers is not critical as long as they are water-soluble. Preferred water-soluble polymers (β5) are starch or starch derivatives or polyvinyl alcohol. The water-soluble polymers, preferably synthetic, such as polyvinyl alcohol, can also be used as a grafting base for the monomers to be polymerized. In another preferred embodiment of the process according to the invention, after drying and comminution, the water-soluble or water-absorbent polymers are mixed with the water-soluble polymers (β5) described above, it being possible for the mixing units known to the expert to be used for the mixing.

In a preferred embodiment of the process according to the invention, the α,β-unsaturated carboxylic acid contained in the liquid phase obtainable by the process according to the invention for the oxidation of unsaturated hydrocarbons, the monomers (β1) and (β2), the crosslinking agents (β3), the adjuvants (β4) and the water-soluble polymers (β5) are employed in an amount such that the water-soluble or water-absorbent polymer obtainable by the process is based on
(γ1) from about 0.1 to about 99.999 wt. %, preferably from about 20 to about 98.99 wt. % and particularly preferably from about 30 to about 98.95 wt. % of monomers
(γ1) or of the α,β-unsaturated carboxylic acid or mixtures thereof,
(γ2) from 0 to about 70 wt. %, preferably from about 1 to about 60 wt. % and particularly preferably from about 1 to about 40 wt. % of the monomers (β2),
(γ3) from about 0.001 to about 10 wt. %, preferably from about 0.01 to about 7 wt. % and particularly preferably from about 0.05 to about 5 wt. % of the crosslinking agents (β3),
(γ4) from 0 to about 20 wt. %, preferably from about 0.01 to about 7 wt. % and particularly preferably from about 0.05 to about 5 wt. % of the adjuvants (β4) and
(γ4) from 0 to about 30 wt. %, preferably from about 1 to about 20 wt. % and particularly preferably from about 5 to about 10 wt. % of the water-soluble polymers (β5), the sum of the amounts by weight (γ1) to (γ5) being about 100 wt. %.

In another preferred embodiment of the process according to the invention, the α,β-unsaturated carboxylic acid, the monomers (β1) and (β2), the crosslinking agents (β3), the adjuvants (β4) and the water-soluble polymers (β5) are employed in an amount such that the water-soluble or water-absorbent polymer comprises to the extent of at least about 50 wt. %, preferably to the extent of at least about 70 wt. % and more preferably to the extent of at least about 90 wt. % monomers which contain carboxylate groups and are based to the extent of at least about 50 wt. %, preferably to the extent of at least about 70 wt. % and more preferably to the extent of at least about 90 wt. %, based on the total weight of the monomers containing carboxylate groups, on those α,β-unsaturated carboxylic acids which were obtained before the polymerization as oxidized hydrocarbons in the liquid phase obtainable by the process according to the invention for the oxidation of unsaturated hydrocarbons. In this connection it is particularly preferable for the water-soluble or water-absorbent polymer to comprise to the extent of at least about 50 wt. %, preferably to the extent of at least about 70 wt. % acrylic acid which is based to the extent of at least about 50 wt. %, preferably to the extent of at least about 70 wt. % and more preferably to the extent of at least about 90 wt. %, based on the total weight of the acrylic acid, on that acrylic acid which was obtained before the polymerization as the oxidized hydrocarbon in the liquid phase obtainable by the process according to the invention for the oxidation of unsaturated hydrocarbons, the acrylic acid preferably being neutralized to the extent of at least about 20 mol %, particularly preferably to the extent of at least about 50 mol %.

In another preferred embodiment of the process according to the invention, the α,β-unsaturated carboxylic acid, the monomers (β1) and (β2), the crosslinking agents (β3), the adjuvants (β4) and the water-soluble polymers (β5) are employed in an amount such that the free acid groups predominate in the polymer formed, so that this polymer has a pH which lies in the acidic range. These acidic water-absorbent polymers can be at least partly neutralized by a polymer with free basic groups, preferably amine groups, which is basic in comparison with the acidic polymer. These polymers are called “mixed-bed ion-exchange absorbent polymers” (MBIEA polymers) in the literature and are disclosed, inter alia, in U.S. Pat. Nos. 6,380,456, 6,258,996, and 6,232,520. As a rule, MBIEA polymers are a composition which comprises on the one hand basic polymers which are capable of exchanging anions and on the other hand a polymer which is acidic compared with the basic polymer and is capable of exchanging cations. The basic polymer contains basic groups and is typically obtained by the polymerization of monomers which carry basic groups or groups which can be converted into basic groups. These monomers are, above all, those which contain primary, secondary or tertiary amines or the corresponding phosphines or at least two of the above functional groups. This group of monomers includes, in particular, ethylene-amine, allylamine, diallylamine, 4-aminobutene, alkyloxycyclines, vinylformamide, 5-aminopentene, carbodiimide, formaldacin, melanine and the like, and secondary or tertiary amine derivatives thereof.

It is furthermore preferable for the liquid phase to contain the α,β-unsaturated carboxylic acid in an amount in a range from about 5 to about 50 wt. %, preferably in a range from about 10 to about 40 wt. % and moreover preferably in a range from about 20 to about 30 wt. %, in each case based on the total weight of the liquid phase. If the liquid phase obtainable by the process according to the invention for the oxidation of unsaturated hydrocarbons contains the α,β-unsaturated carboxylic acid in an amount which lies outside the range described above, the liquid phase can optionally be diluted by addition of water or concentrated before the polymerization, the concentration preferably being carried out by distillation.

It is furthermore preferable in the process according to the invention for the preparation of water-soluble or water-absorbent polymers for the palladium complex to be separated off from the liquid phase containing the α,β-unsaturated carboxylic acids, which was obtained by the process according to the invention for the oxidation of unsaturated hydrocarbons, before the polymerization. The palladium complex is preferably separated off here by filtration of the liquid phase or by chromatographic purification steps, filtration of the liquid phase being particularly preferred.

In one embodiment of the process according to the invention for the preparation of water-soluble or water-absorbent polymers, the α,β-unsaturated carboxylic acid contained in the liquid phase is not concentrated before the polymerization. In another embodiment of the process according to the invention for the preparation of water-soluble or water-absorbent polymers, the liquid phase is employed in an untreated form for the preparation according to the invention of the water-soluble or water-absorbent polymers.

In another embodiment of the process according to the invention for the preparation of water-soluble or water-absorbent polymers, the outer region of the polymer is brought into contact with a crosslinking agent, after drying and comminution of the polymers, so that preferably as a result of which the outer region has a higher degree of crosslinking than the inner region, so that a core-shell structure preferably forms. In this connection it is furthermore preferable for the inner region to have a larger diameter than the outer region. Preferred crosslinking agents (so-called after-crosslinking agents) here are the crosslinking agents of crosslinking agent classes II and IV. Ethylene carbonate is particularly preferred as the after-crosslinking agent.

The invention also relates to the water-soluble or water-absorbent polymers obtainable by the process according to the invention for the preparation of water-soluble or water-absorbent polymers.

In a preferred embodiment of the water-absorbent polymers, these have at least one of the following properties
(A) maximum uptake of about 0.9 wt. % aqueous NaCl in accordance with ERT 440.1-99 in a range from about 10 to about 1,000, preferably from about 15 to about 500 and particularly preferably from about 20 to about 300 ml/g,
(B) the content extractable with about 0.9 wt. % aqueous NaCl solution in accordance with ERT 470.1-99 is less than about 30, preferably less than about 20 and particularly preferably less than about 10 wt. %, based on the untreated absorbent polymer structures, and
(C) the swelling time to achieve about 80% of the maximum absorption of about 0.9 wt. % aqueous NaCl in accordance with ERT 440.1-99 is in the range from about 0.01 to about 180, preferably from about 0.01 to about 150 and particularly preferably from about 0.01 to about 100 min,
(D) the bulk density in accordance with ERT 460.1-99 is in the range from about 300 to about 1,000, preferably from about 310 to about 800 and particularly preferably from about 320 to about 700 g/l,
(E) the pH in accordance with ERT 400.1-99 of about 1 g of the untreated absorbent polymer structure in 1 l of water is in the range from about 4 to about 10, preferably from about 5 to about 9 and particularly preferably from about 5.5 to about 7.5,
(F) CRC in accordance with ERT 441.1-99 in the range from about 10 to about 100, preferably from about 15 to about 80 and particularly preferably from about 20 to about 60 g/g,
(G) AAP in accordance with ERT 442.1-99 under a pressure of about 0.3 psi in the range from about 10 to about 60, preferably from about 15 to about 50 and particularly preferably from about 20 to about 40 g/g.

The combinations of properties of two or more of these properties resulting from the above properties are in each case preferred embodiments of the water-absorbent polymer according to the invention. Embodiments according to the invention which are particularly preferred are furthermore polymers which have the properties or combinations of properties shown below as letters or combinations of letters: A, B, C, D, E, F, G, AB, ABC, ABCD, ABCDE, ABCDEF, ABCDEFG, BC, BCD, BCDE, BCDEF, BCDEFG, CD, CDE, CDEF, CDEFG, DE, DEF, DEFG, EF, EFG, FG.

The invention also relates to the use of a liquid phase containing an α,β-unsaturated carboxylic acid, preferably an aqueous acrylic acid solution, obtainable by the process according to the invention for the oxidation of unsaturated hydrocarbons, for the preparation of water-soluble or water-absorbent polymers.

The invention furthermore relates to the use of a composite comprising a water-absorbent polymer obtainable by the process according to the invention for the preparation of water-absorbent polymers and a substrate. It is preferable for the water-absorbent polymer and the substrate to be firmly bonded to one another. Preferred substrates are films of polymers, such as, for example, of polyethylene, polypropylene or polyamide, metals, nonwovens, fluff, tissues, woven fabric, naturally occurring or synthetic fibres or other foams.

Sealing materials, cable, absorbent cores and diapers and hygiene articles containing these are preferred according to the invention as the composite.

The sealing materials are, preferably, water-absorbent films, wherein the water-absorbent polymer according to the invention is incorporated into a polymer matrix or fibre matrix as the substrate. This is preferably carried out by mixing the water-absorbent polymer with a polymer (Pm) which forms the polymer matrix or fibre matrix and then bonding them by heat treatment if appropriate. In the case where the absorbent structure is employed as a fibre, yarns can be obtained therefrom, which are spun with further fibres made of another material as the substrate and are then bonded to one another, for example by weaving or knitting, or are bonded directly, i.e. without being spun with further fibres. Typical processes for this purpose are described in H. Savano et al., International Wire & Cabel Symposium Proceedings 40, 333 to 338 (1991); M. Fukuma et al., International Wire & Cabel Symposium Proceedings, 36, 350 to 355 (1987) and in U.S. Pat. No. 4,703,132.

In the embodiment in which the composite is a cable, the water-absorbent polymer according to the invention can be employed as particles directly, preferably under the insulation of the cable. In another embodiment of the cable the water-absorbent polymer can be employed in the form of swellable yarns with tensile strength. According to another embodiment of the cable the water-absorbent polymer can be employed as a swellable film. In another embodiment of the cable again, the water-absorbent polymer can be employed as a moisture-absorbing core in the centre of the cable. In the case of the cable, the substrate forms all the constituents of the cable which contain no water-absorbent polymer. These include the conductors incorporated in the cable, such as electrical conductors or light conductors, optical or electrical insulating agents and constituents of the cable which ensure resistance of the cable to mechanical stresses, such as braiding, woven fabric or knitted fabric of material of tensile strength, such as plastics and insulations of rubber or other materials which prevent destruction of the outer skin of the cable.

If the composite is an absorbent core, the water-absorbent polymer according to the invention is incorporated into a substrate. Possible substrates for the cores are chiefly preferably fibrous materials comprising cellulose. In one embodiment of the core the water-absorbent polymer is incorporated in an amount in the range from about 10 to about 90, preferably from about 20 to about 80 and particularly preferably from about 40 to about 70 wt. %, based on the core. In one embodiment of the core the water-absorbent polymer is incorporated into the core as particles. In another embodiment of the core the water-absorbent polymer is incorporated into the core as fibres. The core can be produced on the one hand by a so-called airlaid process or by a so-called wetlaid process, a core produced by the airlaid process being preferred. In the wetlaid process the fibres or particles of water-absorbent polymer are processed to a nonwoven together with further substrate fibres and a liquid. In the airlaid process the fibres or particles of water-absorbent polymer and the substrate fibres are processed to a nonwoven in the dry state. Further details are described in U.S. Pat. No. 5,916,670 and U.S. Pat. No. 5,866,242 for the airlaid process and in U.S. Pat. No. 5,300,192 for the wetlaid process.

In the wetlaid and airlaid process, in addition to the water-absorbent polymer fibres or particles and the substrate fibres further suitable auxiliary substances known to the expert which contribute towards consolidation of the nonwoven obtained from this process can also be added.

In the embodiment in which the composite is a diaper, the constituents of the diaper which differ from the water-absorbent polymer according to the invention represent the substrate of the composite. In a preferred embodiment the diaper comprises a core described above. In this case the constituents of the diaper which differ from the core represent the substrate of the composite. In general a composite employed as a diaper comprises a water-impermeable under-layer, a water-permeable, preferably hydrophobic upper layer, and a layer which comprises the water-absorbent polymer and is arranged between the under-layer and the upper layer. This layer comprising the water-absorbent polymer is preferably a core described above. The under-layer can contain all materials known to the expert, polyethylene or polypropylene being preferred. The upper layer can likewise contain all suitable materials known to the expert, polyesters, polyolefins, viscose and the like being preferred, these resulting in such a porous layer as to ensure an adequate liquid-permeability of the upper layer. The disclosure in U.S. Pat. No. 5,061,295, U.S. Re. 26,151, U.S. Pat. No. 3,592,194, U.S. Pat. No. 3,489,148 and U.S. Pat. No. 3,860,003 is referred to in this connection.

The invention furthermore relates to a process for the production of a composite, wherein a water-absorbent polymer according to the invention and a substrate and optionally a suitable auxiliary substance are brought into contact with one another. They are preferably brought into contact by the wetlaid and airlaid process, compacting, extrusion and mixing.

The invention also relates to a composite obtainable by the above process.

The invention furthermore relates to chemical products, preferably foams, shaped articles, fibres, foils, films, cable, sealing materials, liquid-absorbing hygiene articles, carriers for plant and fungus growth-regulating compositions, additives for building materials, packaging materials and soil additives, which comprise the water-absorbent polymer according to the invention or the composite described above.

The invention also relates to the use of the water-absorbent polymer according to the invention or of the composite described above in chemical products, preferably in foams, shaped articles, fibres, foils, films, cables, sealing materials, liquid-absorbing hygiene articles, carriers for plant and fungus growth-regulating compositions, additives for building materials, packaging materials, for controlled release of active compounds or in soil additives.

According to one embodiment according to the invention of the process according to the invention, the oxidized hydrocarbons according to the invention, the polymers according to the invention and the uses according to the invention, it is preferable for the values of features according to the invention stated only with a lower limit to have an upper limit which has about 20 times, preferably about 10 times and particularly preferably about 5 times the most preferred value of the lower limit.

The invention will now be explained in more detail with the aid of test methods and non-limiting examples.

Test Methods

Gas Chromatography Analysis of Products in the Gas Phase

The gas chromatography analysis of products in the gas phase was carried out with a Shimazu GC 14b gas chromatograph with a flame ionization detector and thermal conductivity detector. The gas phase to be analysed substantially comprised the gases propylene, O₂, N₂, CO₂, CO and the volatile components of the liquid phase. Optimum separation of the individual gaseous components was rendered possible by the following combination of apparatus parameters:

| | |
|---|---|
| Separating | Porapak ® Q from SUPELCO, Bellefonte, PA, |
| columns | USA (external diameter: ⅛ inch, length of the |
| | column for pre-separation: 0.4 m, length of the |
| | Porapak ® Q column: 2.0 m, 80/100 mesh) |
| Main column | Carboxen 1000 from CS- |
| | CHROMATOGRAPHIE-SERVICE GmbH, |
| | Langerwehe (external diameter: ⅛ inch, length |
| | of the column: 5 m, 80/100 mesh) |
| Carrier gas | Helium |
| Carrier gas | 30 ml/min |
| flow rate | |
| Volume of | 100 μl |
| the sample | |
| loop | |
| Temperature | 8 min at 35° C., heated to 160° C. at 15° C./min, then |
| programme | kept at 160° C. for 4.7 min. |

Gas Chromatography Analysis of Products in the Liquid Phase

The analysis of the liquid phase was carried out with an HP 5890 series II gas chromatograph equipped with an FFAP capillary column from J&W SCIENTIFIC, Palo Alto, Calif., USA. Cyclohexanone was used as the standard. The FFAP column had the following features: DB-FFAP, narrow bore, internal diameter 0.25 mm, length 30 m, film 0.25 μm.

Determination of the Propylene Conversion

At the end of the oxidation reaction the amount of unreacted propylene in the gas space (propylene(gas)) is determined by means of gas chromatography analysis. The propylene conversion [%] is defined as follows:

$$propylene conversion [ \% ] = 100 \times \{ \frac{\begin{matrix}propylene ( in ) [ mmol ] - \\ propylene ( gas ) [ mmol ]\end{matrix}}{propylene ( in ) [ mol ]} \}$$

In this equation, propylene(in) is the molar amount of propylene employed at the start.

Determination of the Selectivity of the Oxidation Reaction

At the end of the oxidation reaction the amount of the individual oxidation products in the gas space or in the liquid phase is determined by means of gas chromatography analysis. The amount of propylene reacted results from the propylene conversion defined above. The selectivity [%] is defined as follows:

$$\text{selectivity [\%]} = 100 \times \{ \frac{\text{amount of component in question [mmol]}}{\text{amount of propylene (reacted) [mmol]}} \}$$

Determination of the SCO Value

At the end of the oxidation reaction the amount of the individual oxidation products in the gas space or in the liquid phase is determined by means of gas chromatography analysis. The SCO value of the palladium complex in respect of the individual oxidation products is defined as follows:

$$SCO value [ g \text{/} {g}_{Pd} \text{/} h ] = 100 \times \{ \frac{\text{amount of component in question [g]}}{\text{amount of palladium [g]} \cdot \text{time [h]}} \}$$

### EXAMPLES

To avoid the preparation of highly explosive mixtures in the autoclave for safety reasons, the relative content of propylene and oxygen varies according to the choice of solvent employed. A high molar content of propylene compared with air is used if diglyme or a mixture of water and diglyme is employed, since propylene is very readily soluble in diglyme.

In general, the autoclave is charged with an amount of propylene such that the pressure inside the autoclave is about 4.5 bar. Air is then fed in until a total pressure of about 18 bar is established inside the autoclave. The reaction is preferably carried out at a temperature of about 80° C. The results of experiments 1 to 8 and 9 to 12 are shown in tables 1 and 2.

All the compounds employed in the following examples originate from Acros, Belgium, unless stated otherwise.

### Example 1

100 ml of a 1:1 mixture of water and diglyme is used as the liquid phase. 0.167 g Pd(O₂CCF₃)₂ (0.5 mmol) and 0.053 g solid o-phenanthroline are dissolved in the water/diglyme mixture and a pH of 9 is established with 0.1 N aqueous NaOH solution. The solution obtained in this way is then introduced into an autoclave of rustproof steel with a capacity of 312 ml (stirred autoclave with a heatable jacket and a magnetic coupling for the stirrer from Büchi Glas, Uster; 300 ml, max. 60 bar, max. 220° C.). The autoclave is closed and flushed a few times with helium (purity 99.999%, Messer, Griesheim), with vigorous stirring (Eurostar digital IKA stirrer, 1,000 rpm). 1.71 g (40.7 mmol) propylene and 3.46 g (119.8 mmol) synthetic air (mixture of N₂ (purity 99.999%) and O₂ (purity 99.999%) in a ratio of 79.5:20.5, Messer, Griesheim) are then introduced, a pressure of 17.8 bar being generated inside the autoclave (determined by an electronic pressure sensor from Wika und Setra, Klingenberg). The reactor is then heated up to a temperature of 80° C. (determined by a Haake® DC50/B3 thermostat with external temperature control by means of a Pt-100 thermocouple and a silicone oil bath). After 180 min the gas phase is let out and transferred into a 10 l gas bag (Linde, Wiesbaden) in order to stop the reaction, during which the temperature in the reactor is kept at 80° C. The autoclave is flushed a few times with helium in order to collect the oxygen and unreacted propylene dissolved in the liquid phase. The helium which has been used for the flushing is also transferred into the gas bag. The autoclave is then allowed to cool to room temperature and the liquid phase is removed. The autoclave is then washed out with water and this wash water is combined with the aqueous phase. Both the aqueous phase diluted with the wash water and the gas phase are then analysed by gas chromatography. The selectivity of the reaction is shown in table 1.

### Example 2

The procedure of example 1 is repeated, 0.083 g Pd(O₂CCF₃)₂ (0.25 mmol) and 0.134 g solid bathophen-SO₃ (0.25 mmol) being employed as the catalyst in this experiment. The pH is brought to 8.4 and the reaction is stopped after 120 minutes. The selectivity of the reaction is shown in table 1.

### Example 3

The procedure of example 2 is repeated, exclusively water being used as the liquid phase in this experiment. The pH is brought to 9 and the reaction is stopped after 180 minutes. The selectivity of the reaction is shown in table 1.

### Example 4

The procedure of example 1 is repeated, 0.083 g Pd(O₂CCF₃)₂ (0.25 mmol) and 0.039 g solid 2,2′-dipyridyl (0.25 mmol) being employed as the catalyst in this experiment. The pH is brought to 3.4 and the reaction is ended after 180 minutes. The selectivity of the reaction is shown in table 1.

TABLE 1

| | Duration | Propylene | SCO value for the | Selectivity of the |
|---|---|---|---|---|
| | [min]/T | conversion | acetone synthesis | acetone synthesis |
| Example | [° C.] | [%] | [g/g_{Pd}/h] | [%] |
| 1 | 180/80 | 59 | 4.21 | 64 |
| 2 | 120/80 | 70 | 17.5 | 83 |
| 3 | 180/80 | 39 | 11.4 | 72 |
| 4 | 180/80 | 62 | 21.6 | 68 |

### Example 5 (Comparison)

The procedure of example 1 is repeated, 100 ml water as the liquid phase and 0.114 g Pd(O₂CCH₃)₂ (0.5 mmol) as the catalyst being employed in this experiment. After flushing with nitrogen, 1.71 g (40.7 mmol) propylene and 3.46 g (119.8 mmol) air are added, a pressure of 17.8 bar being obtained inside the autoclave. The pH was brought to 4. The reaction was carried out at a temperature of 80° C. and was stopped after 181 minutes. The selectivity of the reaction is shown in table 2.

### Example 6

The procedure of example 1 is repeated, 100 ml water as the liquid phase and 0.167 g Pd(O₂CCF₃)₂ (0.5 mmol) as the catalyst being employed in this experiment. After flushing with nitrogen, 1.71 g (40.7 mmol) propylene and 3.46 g (119.8 mmol) air are added, a pressure of 17.8 bar being obtained inside the autoclave. The pH was brought to 3.5. The reaction was carried out at a temperature of 80° C. and was stopped after 192 minutes. The selectivity of the reaction is shown in table 2.

### Example 7 (Comparison)

The procedure of example 1 is repeated, 100 ml diglyme as the liquid phase and 0.167 g Pd(O₂CCF₃)₂ (0.5 mmol) as the catalyst being employed in this experiment. After flushing with nitrogen, 8.11 g (192.7 mmol) propylene and 3.01 g (104.4 mmol) air are added, a pressure of 18 bar being obtained inside the autoclave. The autoclave was heated to a temperature of 80° C. After 83 minutes no reaction was to be observed.

### Example 8

The procedure of example 1 is repeated, 100 ml of a 1:1 mixture (based on the particular volume) of water and diglyme as the liquid phase and 0.167 g Pd(O₂CCF₃)₂ (0.5 mmol) as the catalyst being employed in this experiment. After flushing with nitrogen, 2.23 g (53.4 mmol) propylene and 3.46 g (119.8 mmol) air are added, a pressure of 18 bar being obtained inside the autoclave. The pH was brought to 3.5. The reaction was carried out at a temperature of 80° C. and was stopped after 190 minutes. The selectivity of the reaction is shown in table 2.

### Example 9

The procedure of example 1 is repeated, 100 ml of a 3:1 mixture of water and diglyme (based on the particular volume) as the liquid phase and 0.167 g Pd(O₂CCF₃)₂ (0.5 mmol) as the catalyst being employed in this experiment. After flushing with nitrogen, 2.09 g (49.7 mmol) propylene and 3.42 g (118.6 mmol) air are added, a pressure of 18.2 bar being obtained inside the autoclave. The pH was brought to 3.5. The reaction was carried out at a temperature of 80° C. and was stopped after 172 minutes. The selectivity of the reaction is shown in table 2.

### Example 10

The procedure of example 1 is repeated, in this experiment 100 ml water and 0.939 g (7 mmol) diglyme being employed as the liquid phase and 0.167 g Pd(O₂CCF₃)₂ (0.5 mmol) as the catalyst. After flushing with nitrogen, 1.93 g (45.9 mmol) propylene and 3.38 g (116.8 mmol) air are added, a pressure of 18.1 bar being obtained inside the autoclave. The pH was brought to 3.2. The reaction was carried out at a temperature of 80° C. and was stopped after 173 minutes. The selectivity of the reaction is shown in table 2. It can be seen from the results of this experiment that even small amounts of diglyme in the liquid phase allow a selective oxidation of propylene to acrylic acid.

### Example 11

The procedure of example 1 is repeated, 100 ml of a 1:1 mixture (based on the particular volume) of water and diglyme as the liquid phase and 0.167 g Pd(O₂CCF₃)₂ (0.5 mmol) as the catalyst being employed in this experiment. After flushing with nitrogen, 2.10 g (49.4 mmol) propylene and 3.43 g (119.0 mmol) air are added, a pressure of 17.7 bar being obtained inside the autoclave. The pH was brought to 7.5. The reaction was carried out at a temperature of 60° C. and was stopped after 170 minutes. The selectivity of the reaction is shown in table 2.

### Example 12

The procedure of example 1 is repeated, 100 ml of a 1:1 mixture (based on the particular volume) of water and diglyme as the liquid phase and 0.167 g Pd(O₂CCF₃)₂ (0.5 mmol) as the catalyst being employed in this experiment. 0.5 mmol sodium acetate was additionally added. After flushing with nitrogen, 2.26 g (53.7 mmol) propylene and 3.43 g (119.0 mmol) air are added, a pressure of 18 bar being obtained inside the autoclave. The pH was brought to 3.6. The reaction was carried out at a temperature of 100° C. and was stopped after 150 minutes. The selectivity of the reaction is shown in table 2. It can be seen from the comparison of the results of experiments 8 and 12 that the addition of sodium acetate increases the catalytic useful value of the palladium complex containing ligands of the formula (I) and the selectivity of the oxidation of propylene to acrylic acid.

TABLE 2

| | | | SCO value for | Selectivity of the |
|---|---|---|---|---|
| | Duration | Propylene | the acrylic acid | acrylic acid |
| | [min]/T | conversion | synthesis | synthesis |
| Example | [° C.] | [%] | [g/g_{Pd}/h] | [%] |
| 5 | 181/80 | 17.1 | 0.2 | 6.1 |
| 6 | 192/80 | 22.4 | 1.2 | 31.8 |
| 7 | 83/80 | — | — | — |
| 8 | 190/80 | 24.2 | 2.9 | 53 |
| 9 | 172/80 | 26.2 | 2.9 | 48 |
| 10 | 173/80 | 25.9 | 1.6 | 29 |
| 11 | 170/60 | 21.6 | 1.6 | 30 |
| 12 | 150/100 | 28.3 | 3.2 | 40 |

1. A process for the oxidation of unsaturated hydrocarbons, wherein an unsaturated hydrocarbon, an oxygen-containing oxidizing agent, a palladium complex as the catalyst containing a ligand of the formula (I) 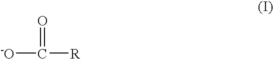
wherein R is a saturated, halogenated alkyl radical having from about 1 to 20 carbon atoms, wherein the palladium complex contains, in addition to the ligand of the formula (I), an organic ligand (X∩Y) which contains at least two atoms X and Y of main group III, V or VI of the periodic table, wherein this ligand can be coordinated to palladium via at least one of these two atoms X and Y and wherein at least one of these atoms is a constituent of a heterocyclic, aromatic ring system, and optionally auxiliary substances are brought into contact with one another
in a liquid phase based on
(α1) from about 10 to about 100 wt. % of a protic polar solvent and
(α2) from 0 to about 90 wt. % of an aprotic polar solvent, the sum of components (α1) and (α2) being about 100 wt. %,
at a temperature in a range from about 30 to about 300° C. under a pressure in a range from about 1 to about 200 bar, such that a liquid phase containing oxygen-containing hydrocarbons is obtained.

2. A process for the oxidation of unsaturated hydrocarbons, wherein an unsaturated hydrocarbon, an oxygen-containing oxidizing agent, a palladium complex as the catalyst containing a ligand of the formula (I) 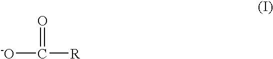
wherein R is a saturated, halogenated alkyl radical having from about 1 to about 20 carbon atoms, and optionally auxiliary substances are brought into contact with one another
in a liquid phase based on
(α1) from about 40 to about 90 wt. % of a protic polar solvent and
(α2) from about 10 to about 60 wt. % of an aprotic polar solvent selected from the group consisting of polyethylene glycol dialkyl ethers, polyethylene glycol divinyl ethers and polyethylene glycol vinyl alkyl ethers, the sum of components (α1) and (α2) being about 100 wt. %,
at a temperature in a range from about 30 to about 300° C. under a pressure in a range from about 1 to about 200 bar, such that a liquid phase containing oxygen-containing hydrocarbons is obtained.

3. A process for the oxidation of unsaturated hydrocarbons, wherein an unsaturated hydrocarbon, an oxygen-containing oxidizing agent, a palladium complex as the catalyst containing a ligand of the formula (I) 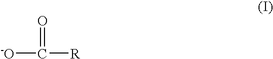
wherein R is a saturated, halogenated alkyl radical having from about 1 to about 20 carbon atoms, and optionally auxiliary substances are brought into contact with one another
in a liquid phase based on
(α1) a protic polar solvent and
(α2) an aprotic polar solvent, the weight ratio of the protic to the aprotic solvent being in a range from about 100,000:1 to about 1:10,
at a temperature in a range from about 30 to about 300° C. under a pressure in a range from about 1 to about 200 bar, such that a liquid phase containing oxygen-containing hydrocarbons is obtained, the protic polar solvent not being water and the aprotic polar solvent not being diglyme.

4. A process for the oxidation of unsaturated hydrocarbons, wherein an unsaturated hydrocarbon, an oxygen-containing oxidizing agent, a palladium complex as the catalyst containing a ligand of the formula (I) 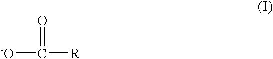
wherein R is a saturated, halogenated alkyl radical having from about 1 to about 20 carbon atoms, and optionally auxiliary substances are brought into contact with one another
in a liquid phase based on
(α1) water and
(α2) diglyme, the weight ratio of the water to the diglyme being in a range from about 100,000:1 to about 1:10,
at a temperature in a range from about 30 to about 300° C. under a pressure in a range from about 1 to about 200 bar, such that a liquid phase containing oxygen-containing hydrocarbons is obtained.

5. The process according to claim 1, wherein the radical R is a trifluoromethyl radical.

6. The process according to claim 1, wherein the oxygen-containing oxidizing agent is chosen from the group consisting of O₂, H₂O₂ and N₂O.

7. The process according to claim 1, wherein the liquid phase is a mixture of water and diglyme.

8. The process according to claim 1, wherein the unsaturated hydrocarbon is propylene.

9. The process according to claim 1, wherein the palladium complex is first activated by reduction before it catalyses the oxidation of the unsaturated hydrocarbon.

10. The process according to claim 2, wherein the palladium complex contains, in addition to the ligand of the formula (I), an organic ligand (X∩Y) which contains at least two atoms X and Y of main group III, V or VI of the periodic table, wherein this ligand can be coordinated to palladium via at least one of these two atoms X and Y and wherein at least one of these atoms is a constituent of a heterocyclic, aromatic ring system.

11. The process according to claim 1, wherein the organic ligand (X∩Y) can be coordinated to palladium as a bidentate ligand via the two atoms X and Y.

12. The process according to claim 1, wherein the organic ligand (X∩Y) is selected from the group consisting of p-bathophen-sulfonate and 2,2′-bipyridyl.

13. The process according to claim 1, wherein acetic acid or a salt of acetic acid is employed as the auxiliary substance.

14. The process according to claim 13, wherein the acetic acid or a salt of acetic acid is employed as the auxiliary substance
(δ1) to increase the catalytic useful value of the palladium complex in the oxidation of unsaturated hydrocarbons, or
(δ2) to increase the selectivity of the oxidation of unsaturated hydrocarbons.

15. A process for the preparation of water-soluble or water-absorbent polymers, wherein, in a liquid phase obtained by a process for the oxidation of propylene, wherein propylene, an oxygen-containing oxidizing agent, a palladium complex as the catalyst containing a ligand of the formula (I) 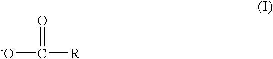
wherein R is a saturated, halogenated alkyl radical having 1 to 20 carbon atoms, and optionally auxiliaries are brought into contact with one another in a liquid phase based on
(α1) from about 10 to about 100 wt. % of a protic polar solvent and
(α2) from 0 to about 90 wt. % of an aprotic polar solvent, the sum of components (α1) and (α2) being about 100 wt. %,
at a temperature in a range from about 30 to about 300° C. under a pressure in a range from about 1 to about 200 bar, the acrylic acid contained as the oxygen-containing hydrocarbon is polymerized and the water-soluble or water-absorbent polymer obtained in this way is then optionally dried and comminuted.
